(19) United States
(12) Patent Application Publication
(10) Pub. No.: US 2025/0299772 A1
(43) Pub. Date: Sep. 25, 2025

(54) METHOD FOR GENERATION OF CHEMICAL DERIVATIVES AGAINST TARGET PROTEIN TO BUILD AI DRUG PLATFORM

(71) Applicant: SYNTEKABIO, INC., Daejeon (KR)

(72) Inventors: Jong Sun JUNG, Daejeon (KR); Jong Hui HONG, Seoul (KR); Seung Min YOON, Yongin-si Gyeonggi-do (KR); Bo Su KIM, Seoul (KR)

(73) Assignee: SYNTEKABIO, INC., Daejeon (KR)

(21) Appl. No.: 19/226,403

(22) Filed: Jun. 3, 2025

Related U.S. Application Data
(63) Continuation of application No. PCT/KR2024/014763, filed on Sep. 27, 2024.

(30) Foreign Application Priority Data
Mar. 21, 2024 (KR) 10-2024-0039328

(51) Int. Cl. G16B 15/30; G06N 20/00; G16B 40/30

(52) U.S. Cl. CPC G16B 15/30; G06N 20/00; G16B 40/30

(57) ABSTRACT

A method for generating a hit compound derivative from a hit compound for a target protein, the method comprising: (A) selecting a substitutable portion and a scaffold excluding the substitutable portion in a chemical structure of the hit compound; (B) setting a target space within the target protein, around a region where the selected substitutable portion of the hit compound binds; and (C) selecting a substituent that can replace the substitutable portion of the hit compound within the set target space of the target protein, and generating the hit compound derivative.

### CROSS-REFERENCE TO RELATED APPLICATIONS

This application is a bypass continuation application of international application No. PCT/KR2024/014763 having an international filing date of Sep. 27, 2024 and designating the United States, the international application being based upon and claiming the benefit of priority from Korean Patent Application No. 10-2024-0039328, filed on Mar. 21, 2024, the entire contents of which are incorporated herein by reference.

### TECHNICAL FIELD

The present disclosure relates to a method for generation of various forms of derivatives from an existing hit compound (hit compound) against a specific target protein, which is a technology useful for in silico prescreening applicable to the process of discovering drug candidates to build Computer-Aided Drug Discovery (CADD) or artificial intelligence (AI) drug platform for the development of drugs, an in silico derivative generated by the corresponding method, and an AI drug discovery platform built using the method for generating derivatives disclosed herein.

### BACKGROUND

Traditional drug development involves identifying a mechanism or protein to be targeted in relation to a specific disease, discovering and screening candidate compounds that act on the target, and proceeding through optimization of candidate materials, preclinical/toxicological testing, and clinical trials. This conventional drug development process takes an average of five years to identify drug candidates and an additional two years to select candidates for clinical trials. Despite the significant research and development/clinical trial costs and an average development period of 15 years, only about 10% of candidate compounds successfully pass the final approval of regulatory authorities (e.g., the U.S. FDA).

In recent years, computing analysis technologies (such as AI) have been actively utilized in the drug development process to reduce time and cost while increasing final success rates.

AI technology in drug development is used not only for rapid data analysis but also for various purposes such as searching for optimal drug candidates and analyzing suitable clinical trial participants. In the field of computational screening for drug candidate identification and analysis, various analytical tools are used, as exemplified in Table 1 below.

TABLE 1

| | | | Analysis Time |
|---|---|---|---|
| Program Name | Type | Principle | per Pose |
| AutoDock | 3D docking | Grid-based semi- | Minutes to |
| | | empirical scoring | hours |
| | | function (considering van | |
| | | der Waals forces, | |
| | | electrostatic interactions, | |
| | | and desolvation) | |
| Glide | 3D docking | Empirical scoring | 0.2-2.4 minutes |
| | | function (GlideScore) | |
| | | (considering Coulomb, | |
| | | van der Waals, and | |
| | | solvation effects) | |
| GOLD (Genetic | 3D docking | ChemScore-based scoring | Minutes to |
| Optimization | | function (multiple | hours |
| for Ligand Docking) | | binding modes) | |
| FEP+ | Molecular dynamics | Free energy perturbation | Hours to days |
| | | | (depending on |
| | | | system size and |
| | | | settings) |
| PLUMED/GROMACS | Molecular dynamics | Meta-dynamics | Hours to days |
| Gaussian | QM/MM | Quantum | Hours to days |
| | | mechanics/molecular | (depending on |
| | | mechanics | QM region |
| | | | size) |
| AMBER | Molecular dynamics | Alchemical Free Energy | Hours to days |
| | | (e.g., thermodynamic | |
| | | integration) | |
| DeepChem | Deep Learning | Neural networks for | Seconds to |
| | | molecular systems | minutes (once |
| | | | trained) |
| gnina | Deep Learning | 3D convolutional neural | 2.5 minutes |
| | | network-based affinity | (once trained) |
| | | prediction | |
| Phase, Discovery | Pharmacophore | Generates pharmacophore | Minutes to |
| Studio, MOE | modeling | models from known hit | hours |
| | | compounds or protein | |
| | | structures | |
| LigandScout | Pharmacophore | Generates a | Hours |
| | ensemble approach | pharmacophore ensemble | |
| | | from multiple active | |
| | | ligands or protein | |
| | | structures | |
| ROCS | 3D alignment/2D | Rapid overlay of | Minutes to |
| | fingerprint field | chemical structures based | hours |
| | | on shape and chemical | |
| | | features for virtual | |
| | | screening | |

The process of virtually screening drug candidates from a compound library can be divided into (1) a pre-screening stage based on chemical properties or structural similarity and (2) a deep screening stage utilizing 3D docking protein-ligand interaction data. The prescreening stage is typically used to reduce the number of candidate compounds from a large library and employs simple numerical filtering algorithms, such as Lipinski's Rule of Five (guidelines for drug design). Due to the limited information used for screening, the screening reliability is generally maintained at approximately 10%.

However, with the advancement of computing technology, the demand for processing billions of analytes has increased, necessitating the enhancement of traditional screening methods.

To address this necessity, various strategies have been explored, such as comparing and analyzing the similarity of compound features, including chemical properties, or characterizing the two-dimensional structural patterns of molecules. However, screening based on these strategies has limitations, such as a low true-positive (T/P) ratio and the inability to patternize the structure of all substances.

Meanwhile, 3D and 4D-based protein-ligand binding affinity prediction technologies, which are known for their relatively high screening reliability, require analysis times ranging from several minutes to hours per structure. This makes them unsuitable for large-scale screening applications.

As related references,
(Patent Literature 1) Korean Patent No. 10-2496208
(Patent Literature 2) Korean Patent No. 10-0984735
(Patent Literature 3) Korean Patent No. 10-2181058
(Non-Patent Literature 1) Garrett Morris et al. (2009) “AutoDock4 and AutoDockTools4: Automated docking with selective receptor flexibility.” Journal of Computational Chemistry 30(16): 2785-2791
(Non-Patent Literature 2) Richard Friesner et al. (2004) “Glide: A New Approach for Rapid, Accurate Docking and Scoring. 1. Method.” Journal of Medicinal Chemistry 47:1739-1749
(Non-Patent Literature 3) G. Jones et al. (1995) “Molecular recognition of receptor sites using a genetic algorithm with a description of desolvation.” Journal of Molecular Biology 245(1): 43-53
(Non-Patent Literature 4) Daniel Cappel et al. (2016) “Relative Binding Free Energy Calculations Applied to Protein Homology Models.” Journal of Chemical Information and Modeling 56(12): 2388-2400, and
(Non-Patent Literature 5) Alessandro Laio et al. (2002) “Escaping free-energy minima.” Proceedings of the National Academy of Sciences 99(20): 12562-12566 exist as patent and non-patent literatures.

### SUMMARY

### Technical Problem

The present disclosure has been made to solve the above-described problems and provides a derivative generation method capable of improving the efficacy or pharmacokinetic properties of a previously identified or derived active compound (hit compound) and generating various derivatives for training an analysis AI algorithm.

This is achieved by setting a target space within the target protein, focusing on the region where the target protein and the hit compound bind, and generating derivatives of the hit compound that can be accommodated within this space. Additionally, the method may be executed on a computational system.

Through the derivative generation method of the present disclosure, it has been confirmed that in spite of an in silico derivative generation method, the generated derivatives exhibit an enhanced likelihood of actual binding to the target protein. Furthermore, it has been confirmed that the generated derivatives have improved binding affinity with the target protein or exhibit an enhanced effect on the activity of the target protein compared to the hit compound, thereby completing the present disclosure.

### Technical Solution

1. An aspect of the present disclosure may pertain to a method for generating a hit compound derivative from a hit compound for a target protein,
the method including:
(A) selecting a substitutable portion and a scaffold excluding the substitutable portion in a chemical structure of the hit compound;
(B) setting a target space within the target protein, around a region where the selected substitutable portion of the hit compound binds; and
(C) selecting a substituent that can replace the substitutable portion of the hit compound within the set target space of the target protein, and generating the hit compound derivative.

2. In an embodiment, the method may be executed in a computational system.

3. In an embodiment, the method may be for constructing an artificial intelligence (AI)-drug platform.

4. In an embodiment of the method, the substitutable portion in the chemical structure of the hit compound in step (A) may be selected based on an indicator showing lower binding interaction compared to other chemical structures, as determined from an interaction profile between the hit compound and the target protein.

5. In an embodiment, the binding interaction may be determined by: cleaving individual bonds within the hit compound that interact with the target protein, selecting a substitution candidate portion that includes atoms at the cleaved site for each cleaved site, and calculating an average binding energy between each atom constituting the substitution candidate portion and the target protein.

6. In an embodiment, the substitution candidate portion may include 1 to 12 atoms.

7. In an embodiment, the method may further include filtering the substitution candidate portion based on the number of constituent atoms.

8. In an embodiment of the method, a size of the target space within the target protein in step (B) is set to accommodate the selected substitutable portion of the hit compound in the chemical structure of the hit compound binding to the target protein.

9. In an embodiment of the method, the setting in step (B) may include determining the region of the target protein constituting the target space within the target protein by stepwise classification based on interaction energy between atoms of the target protein present in the region.

10. In an embodiment, the interaction energy between atoms of the target protein for classifying regions of the target protein may be classified into three to five levels.

11. In an embodiment, the method may include a step of clustering regions with relatively lower level of the interaction energy and further selecting the clustered regions of the target space to be used for generating the hit compound derivative based on proximity to the scaffold of the hit compound and size of the clustered regions.

12. In an embodiment, the classification of interaction energy between atoms of the target protein in step (B) of the method is performed by: extracting the region of the target protein constituting the target space as a spatial filter, setting marker points (dots) arranged at equal intervals in the spatial filter, and classifying the marker points based on interaction energy between atoms of the target protein.

13. In an embodiment, the spatial filter may be a spherical, rectangular, cylindrical, or amorphous filter.

14. In an embodiment, the selection of clustered regions of the target space to be used for generating the hit compound derivative in step (B) may include:
(B1) extracting the region of the target protein constituting the target space as a cylindrical filter form, setting marker points (dots) arranged at equal intervals in the cylinder filter, and stepwise classifying the marker points based on interaction energy between atoms of the target protein; and
(B2) approaching the scaffold of the hit compound to the cylindrical filter, excluding regions where interaction energy between atoms of the target protein exceeds a preset threshold from the cylinder filter, and then clustering the remaining marker points into spatial units.

15. In an embodiment, the selection of clustered regions of the target space to be used for generating the hit compound derivative in step (B) may further include a step of (B3) selecting a part of the clustered regions as the target space to be used for generating the hit compound derivative, based on proximity between marker points and the scaffold of the hit compound and the size of the clustered regions in addition to steps (B1) and (B2).

16. In an embodiment, the clustering is performed based on the density of regions with relatively lower level of the interaction energy, or by using Gaussian Mixture Model (GMM) clustering.

17. In an embodiment, step (C) may be performed by replacing the substitutable portion that binds to the scaffold of the hit compound, selected in step (A), with a substituent selected from a substituent group database, which can be accommodated within the target space set in step (B).

18. In an embodiment, step (C) may include generating a derivative having multiple different binding conformations for the same substituent by varying the binding position within the substituent that binds to the scaffold of the hit compound.

19. In an embodiment, step (C) may include generating a derivative having multiple different binding pose for substituents with the same binding conformation by varying the binding angle between the substituents and the scaffold of the hit compound.

20. In an embodiment, step (C) may include generating a derivative having multiple different binding poses (single bond, double bond, triple bond, etc.) for the substituent with the same binding conformation by varying the binding between the substituent and the scaffold of the hit compound.

21. In an embodiment,
the substitutable portion in the chemical structure of the hit compound in step (A) may be selected based on an indicator showing lower level of the binding interaction compared to other chemical structures, as determined from the interaction profile between the hit compound and the target protein;
the selection of the target space to be used for generating the hit compound derivative of the target protein in step (B) may be performed by the steps of:
(B1) extracting the region of the target protein constituting the target space as a cylindrical filter form, setting marker points (dots) arranged at equal intervals within the cylindrical filter, and stepwise classifying the marker points based on the interaction energy between atoms of the target protein,
(B2) approaching the scaffold of the hit compound to the cylindrical filter, excluding regions where the interaction energy exceeds a preset threshold from the cylindrical filter, and then clustering the remaining marker points into spatial units, and
(B3) selecting a portion of the clustered regions based on the proximity between marker points (dots) in the clustered regions and the size of the clustered regions; and
the derivative generation in step (C) may be performed by selecting substituents that can be accommodated within the target space selected in step (B).

22. In an embodiment, the method may further include (D) filtering the generated derivatives.

23. In an embodiment, step (D) may include filtering the binding angle between the substituent and the scaffold of the hit compound compared to a real compound database.

24. In an embodiment, step (D) may include filtering the generated derivatives based on degree of atomic clashes between the derivative and the target protein atoms, which occurs within the target space set in the derivatives in step (B), according to the binding pose of the substituents.

25. An aspect of the present disclosure may pertain to an in silico derivative generated by the method for generating a derivative according to an aspect of the present disclosure.

26. An aspect of the present disclosure may pertain to an AI-drug platform constructed by the method for generating a derivative according to an aspect of the present disclosure.

### Advantageous Effects

The method for generating, from a hit compound for a target protein, corresponding hit compound derivatives according to an aspect of the present disclosure enables the improvement of the efficacy or pharmacokinetic properties of a previously identified or derived hit compound and the generation of various derivatives for training an analysis AI algorithm.

Through the derivative generation method according to an aspect of the present disclosure, it is possible to generate derivatives with an enhanced likelihood of actual binding to the target protein.

Through the derivative generation method according to an aspect of the present disclosure, it is possible to generate derivatives that exhibit improved binding affinity for the target protein or an enhanced effect on the activity of the target protein compared to the hit compound.

Through the derivative generation method according to an aspect of the present disclosure, as the binding poses of compounds that can bind within the target space are derived along with the generation of derivatives, when molecular dynamics simulations are performed in an artificial intelligence drug platform, the possibility of deriving the optimal binding pose is enhanced.

### DESCRIPTION OF DRAWINGS

FIG. 1 is a configurational diagram illustrating an exemplary overall structure of an artificial intelligence-drug platform (AI-drug platform) constructed by applying the compound derivative generation method for a target protein according to the present disclosure.

FIG. 2 is a conceptual diagram illustrating an exemplary cloud service structure of an AI-drug platform constructed by applying the compound derivative generation method for a target protein according to the present disclosure.

FIG. 3 is a conceptual diagram illustrating the process of discovering hit compounds in the AI-drug platform constructed by applying the compound derivative generation method for a target protein according to the present disclosure.

FIG. 4 is a conceptual diagram illustrating the process of lead compound discovery in the AI-drug platform constructed by applying the compound derivative generation method for a target protein according to the present disclosure.

FIG. 5 is a flowchart illustrating a lead compound discovery method through derivative generation according to a specific embodiment of the compound derivative generation method for a target protein according to the present disclosure.

FIGS. 6A and 6B are conceptual diagrams illustrating a method of selecting a substitutable portion in the process of the compound derivative generation method for a target protein according to the present disclosure, specially including a process of selecting an anchor atom on the scaffold, which is the atom to which the substitutable portion is bound.

FIGS. 7A, 7B, 8A through 8F, and 9 are conceptual diagrams illustrating the process of calculating the size of a target space in the process of the compound derivative generation method for a target protein according to the present disclosure.

FIGS. 10, 11A, and 11B are conceptual diagrams illustrating a method of selecting an R-group (substituent) to generate a derivative in the process of the compound derivative generation method for a target protein according to the present disclosure.

FIGS. 12A and 12B are exemplary diagrams illustrating a process of extracting a linker portion of an anchor region of the target protein in the process of the compound derivative generation method for a target protein according to the present disclosure and filtering based on a comparison between the binding form of the linker portion and the binding form of the substituent with that of an existing compound.

FIGS. 13A and 13B are exemplary diagrams illustrating a process of filtering generated derivatives based on atomic clashes within the target space in the process of the compound derivative generation method for a target protein according to the present disclosure.

FIG. 14 illustrates a step of evaluating the interaction profile, selecting the substitutable portion, and identifying the scaffold portion based on a known hit compound (hit compound X) that is already known to bind to a specific target protein (protein A), according to a specific embodiment of the compound derivative generation method for a target protein according to the present disclosure.

FIG. 15 shows images after a region filter from a target region (also referred to as a pocket region) within the target protein is extracted based on the atom binding to the substitutable portion on the scaffold of the hit compound (that is, anchor atom) and marker points are set at equal intervals (in FIG. 15, total cylinder). A total cylindrical region is used for region extraction. The van der Waals radius (vdW radius; r) of the atoms within the target protein is calculated. Based on the calculated r values, marker points are classified as follows: red dots (Red zone; clash region) for points within approximately 0.7r; yellow dots (yellow zone; buffer region) for points within approximately 0.7r to 1.0r; green dots (green zone; contact region) for points within approximately 1.0r to 1.3r; and gray dots (gray zone) for points beyond approximately 1.3r.

FIG. 16 is a graph illustrating a method of selecting derivatives in the process of the compound derivative generation method for a target protein according to the present disclosure, wherein the substitutable portion is replaced with substituents that can be accommodated within the extracted target space, and the derivatives are ranked in order of low binding energy (e.g., selecting the bottom 5%).

FIG. 17 illustrates a modeled spatial configuration of derivatives expected to have optimal substituents and their binding poses.

### DETAILED DESCRIPTION OF THE INVENTION

Embodiments of the present disclosure are illustrated for describing the technical spirit of the present disclosure. The scope of the claims according to the present disclosure is not limited to the embodiments described below or to the detailed descriptions of these embodiments. Various embodiments or examples described herein are illustrated for the purpose of clearly explaining the technical spirit of the present disclosure, and are not intended to be limited to specific embodiments. The technical idea of the present disclosure includes various modifications, equivalents, substitutes for each embodiment or examples described herein, and embodiments or examples that are selectively combined from all or part of the respective embodiments or examples.

All technical or scientific terms used herein have meanings that are generally understood by a person having ordinary knowledge in the art to which the present disclosure pertains, unless otherwise specified. The terms used herein are selected for only more clear illustration of the present disclosure, and are not intended to limit the scope of claims in accordance with the present disclosure.

A singular expression can include meanings of plurality, unless otherwise mentioned, and the same is applied to a singular expression stated in the claims.

Each block of the flowcharts and/or block diagrams, and combinations of blocks in the flowcharts and/or block diagrams described herein, can be implemented by computer program instructions (execution engine). These computer program instructions may be provided to a processor of a general-purpose computer, special purpose computer, or other programmable data processing apparatus, such that the instructions which are executed via the processor of the computer or other programmable data processing apparatus create means for implementing the functions/acts specified in the flowcharts and/or block diagrams.

Furthermore, the computer program instructions may also be loaded onto a computer or other programmable data processing apparatus to cause a series of operations to be performed on the computer or other programmable apparatus to produce a computer implemented process such that the instructions which are executed on the computer or other programmable apparatus provide operations for implementing the functions/acts specified in the flowcharts and/or block diagrams.

Furthermore, the respective block diagrams may illustrate parts of modules, segments, or codes including at least one or more executable instructions for performing specific logic function(s). In some alternative embodiments, the functions mentioned in the blocks or steps may occur in a different order than described.

Moreover, in the field of technology to which the present disclosure is applied, a majority of technical terms are commonly used with their English designations rather than being defined in Korean. Accordingly, technical terms that are presented with Korean equivalents in the present disclosure should be interpreted based on the meaning of the corresponding English terms that are generally recognized in the technical field.

### I. Definitions

In the present disclosure, the term “about” may represent a conventional margin of error for each value, as widely recognized by those skilled in the art. In the context of numerical values or ranges described in the present disclosure, this may mean+20%, +15%, +10%, +9%, +8%, +7%, +6%, +5%, +4%, +3%, +2%, or +1% of the stated or claimed numerical value or range in an aspect.

The expressions “include”, “provided with”, “have” and the like used herein should be understood as open-ended terms connoting the possibility of inclusion of other embodiments, unless otherwise mentioned in a phrase or sentence including the expressions.

The term “and/or,” as used herein, may mean any one or more of the associated items, any combination of the items, or all of the items.

An aspect of the present disclosure described herein is understood to include embodiments that “comprise,” “consist of,” and/or “consist essentially of” the relevant components.

### II. Components Related to the Method for Generating Derivatives of a Hit Compound in the Present Disclosure

### Configurations Included in Technology of the Present Disclosure

The term “target protein,” as used herein, refers to a protein that binds to a hit compound in the present disclosure and whose characteristics, such as activity or stability, may be affected by the binding with the hit compound. In the present disclosure, derivatives are generated based on the binding properties between the target protein and the hit compound.

As used herein, the term “hit compound” refers to a molecule, compound, or ligand that binds to the target protein in the present disclosure with high affinity. That is, the term is intended to encompass any molecule, ligand, or compound that binds to the target protein in the present disclosure either covalently or non-covalently.

The term “derivative of a hit compound” or “derivative,” as used herein, refer to a compound generated by substituting, deleting, or adding a specific functional group to a portion of the hit compound in the present disclosure. For example, the chemical structure of the hit compound in the present disclosure may be divided into a substitutable portion and a scaffold portion, and a derivative of the hit compound may be generated by replacing the substitutable portion with a different substituent.

The term “substitutable portion,” as used herein, refers to a portion of the chemical structure of the hit compound that binds to the target protein and is selected as a target for substitution. This selection may be based on the binding interactions between the target protein and the hit compound. For example, a portion of the chemical structure within the hit compound may be selected as the substitutable portion if it exhibits lower binding interactions compared to other chemical structures, based on the interaction profile between the target protein and the hit compound. The portion of the hit compound excluding the substitutable portion is designated as the “scaffold”.

The term “interaction profile,” as used herein, refers to an arrangement of information on atomic types, physicochemical properties, and types of interactions. Specifically, the interaction profile may be obtained using commercially available software (FEP+, MMPBSA, and so on). In this regard, conventional DSSP (Dictionary of Protein Secondary Structure) programs based on Coulomb's force and Coulomb's law exist, and calculations may be performed using software based on various algorithms improved from the DSSP program.

The “binding interaction,” as used herein, may be measured by calculating the binding energy of atoms involved in the binding between the target protein and the hit compound. For example, the binding energy may be calculated for each atom involved in the binding between the target protein and the hit compound and may be derived as a median or average value of these energies. Furthermore, to calculate the binding interaction value, each bond within the chemical structure of the hit compound that interacts with the target protein may be sequentially cleaved. For each cleaved bond, a substitution candidate portion may be selected, which includes the atoms or chemical structure portion on the cleaved side that interacts with the target protein. The binding energy between each atom constituting the substitution candidate portion and the target protein may then be calculated and derived as an average value or similar metric. The term “binding interaction,” as used herein, may refer to interaction efficiency, which is commonly used in the relevant technical field, and may be calculated as the average binding energy of the atoms constituting a specific functional group or a specific portion of a compound.

The “interaction efficiency” in the present disclosure may be calculated using the following equation:

$$Interaction Efficiency = \frac{{\sum }_{atom} Δ Energy}{\# atom} \qquad [ Equation ]$$

The term “substitution candidate portion,” as used herein, refers to atoms or functional groups within the hit compound that may serve as candidates before selecting the “substitutable portion” in the present disclosure. This selection process involves sequentially cleaving bonds (e.g., single bonds, double bonds, etc.) within the portion of the hit compound that interacts with the target protein. Two atomic fragments are created by this process, and the portion that is closer to or interacts more strongly with the target protein may be selected as the substitution candidate portion. The substitution candidate portion may include 1 to 12 atoms.

The terms “target space within the target protein,” “target region,” “pocket space,” or “target space,” as used herein, refer to an analysis region set for performing the derivative generation method of the present disclosure. This region is established within the target protein, around the area where the substitutable portion of the hit compound binds. The size of the target space within the target protein may be set to accommodate the substitutable portion of the hit compound selected from the chemical structure of the hit compound that binds to the target protein. For example, the size of the target space within the target protein may be set such that it accommodates: (1) the substitutable portion, and (2) the region excluding the substitutable portion (i.e., the portion of the scaffold of the hit compound that belongs to the binding region) in the chemical structure of the hit compound binding to the target protein. In the present disclosure, the target space may be set to a predetermined size or may be determined to include both the selected substitutable portion and the atoms of the target protein interacting with the substitutable portion based on the binding conformation between the target protein and the hit compound. The region of the target protein, which constitutes the target space thus set, may be modeled in three dimensions. The target protein region constituting the target space within the target protein may be stepwise classified based on the interaction energy of the target protein atoms present in that region. Furthermore, the target space within the target protein may be extracted in the form of a cylindrical filter, and marker points (dots) arranged at equal intervals may be set in the cylindrical filter. These marker points may then be stepwise classified based on the interaction energy of the atoms of the target protein.

The term “clustering,” as used herein, refers to defining a specific space or region as a single area based on identical or similar characteristics within a given space area. In the present disclosure, interaction energy exerted by the atoms of the target protein may be used as an indicator for defining the region. For example, in the present disclosure, after the target protein region constituting the target space in the target protein is classified stepwise based on the interaction energy of the target protein atoms present in the corresponding region, the clustering may be achieved by excluding regions with relatively high interaction energy or leaving only regions with lower interaction energy. The clustering in the present disclosure may be further determined based on the proximity to the scaffold of the hit compound and/or the size of the clustered region. In a specific embodiment, the clustering may be achieved by extracting the target protein region constituting the target space as the form of a cylindrical filter, setting marker points (dots) arranged at equal intervals in the cylindrical filter, stepwise classifying the marker points based on the interaction energy of the target protein atoms, and retaining only the marker points corresponding to relatively low interaction energy. The clustering in the present disclosure may be performed based on the density of regions with relatively low interaction energy or may be conducted using a Gaussian Mixture Model (GMM) clustering method.

The term “computational system,” as used herein, refer to a system that receives data as input, processes the data, and outputs results based on rules or algorithms. The computational system may have the capability to perform operations such as computation, processing, and/or transformation automatically and may include hardware and/or software components. The computational system may include input, processing, and output stages as its components. The input stage is the stage where data is received for solving complex problems through multiple computational processes. Data may be received from a user or automatically acquired from other systems or sensors. The processing stage is the stage where computations are performed through algorithms or programs, processing the received input data. The output stage is the stage where the processed results are output. The output may be displayed on a screen, transmitted to another system, or stored. Examples of a computational system may include a computer, an artificial intelligence (AI) system, and other similar systems.

### III. Aspects of Method for Generating Derivatives of Hit Compound in the Present Disclosure

Before describing the method for generating derivatives of a hit compound for a target protein according to the present disclosure, an explanation of the entire AI drug platform to which the method is applied will be provided.

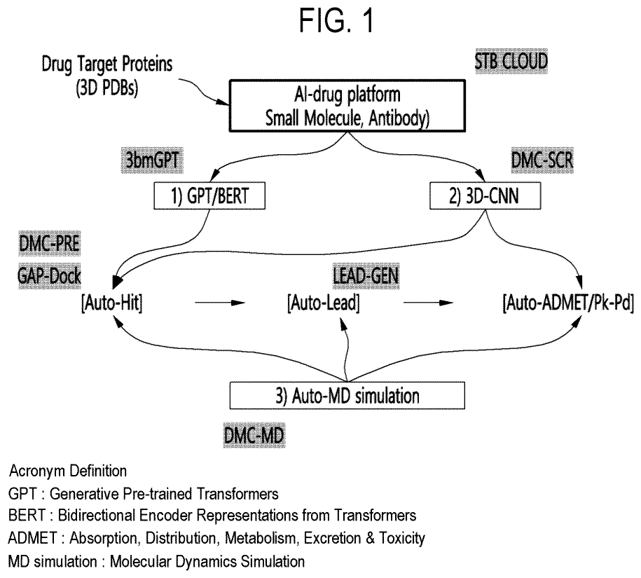

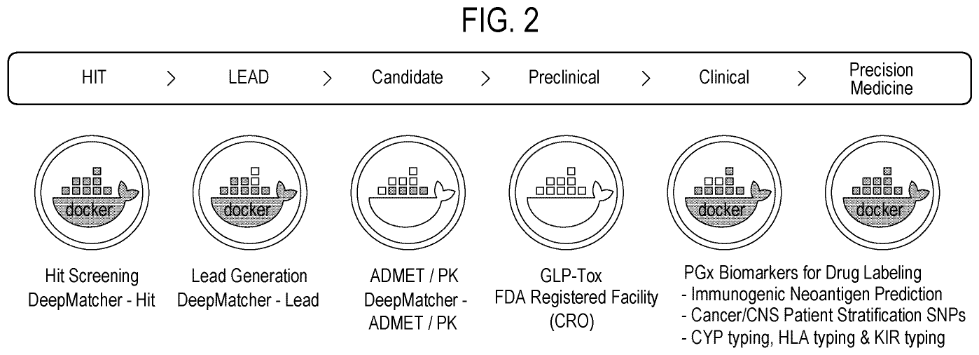

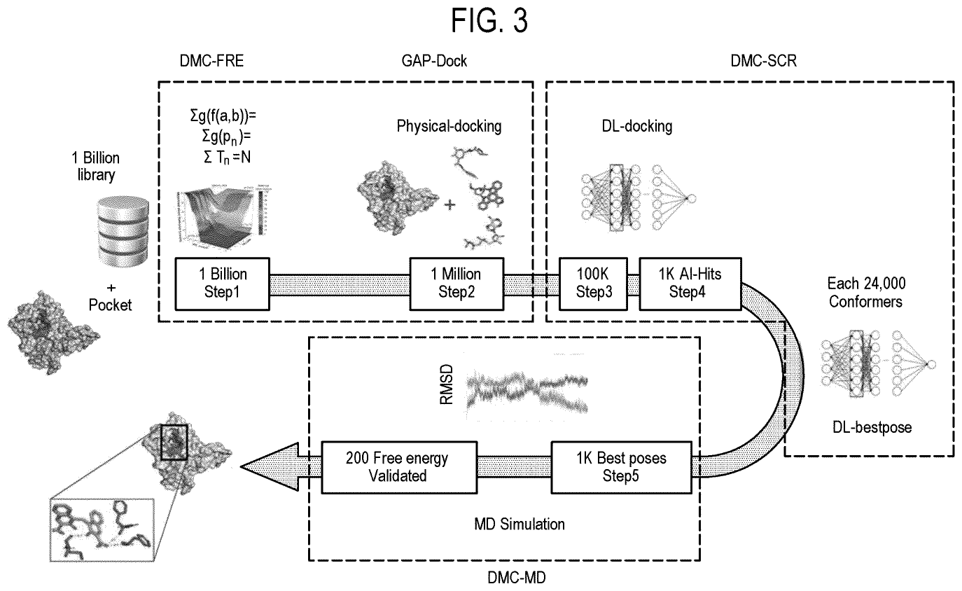

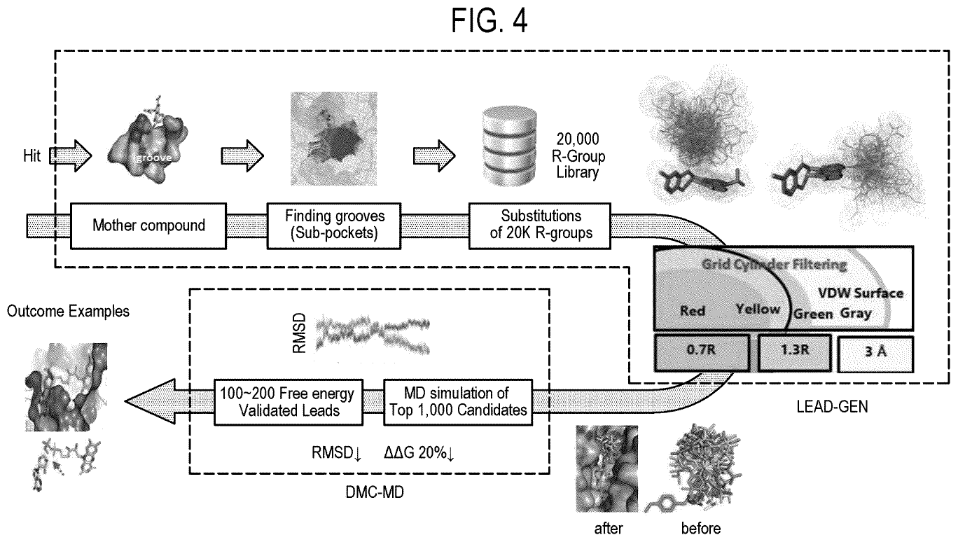

FIG. 1 is a configuration diagram illustrating the overall structure of an AI-drug platform in which the technology of the present disclosure is applied. FIG. 2 is a conceptual diagram illustrating the cloud service structure of the AI-drug platform in which the technology of the present disclosure is applied. FIG. 3 is a conceptual diagram illustrating the hit compound discovery process of the AI-drug platform in which the technology of the present disclosure is applied. FIG. 4 is a conceptual diagram illustrating the lead compound discovery process of the AI-drug platform in which the technology of the present disclosure is applied.

The AI-drug platform in which the technology of the present disclosure is applied is fundamentally a platform that performs the entire process of discovering new drug candidates in the preclinical stage and may be provided as a cloud-based service (STB CLOUD).

In this regard, the new drugs that may be serviced by the AI-drug platform include new synthetic drugs (small molecules) and new antibody drugs. The AI-drug platform according to the present disclosure provides discovery processes for both types of drugs.

To this end, as illustrated in FIG. 1, the AI-drug platform according to the present disclosure comprises an automated active (hit) compound discovery platform, an automated lead compound discovery platform, and an automated drug response (ADMET: Absorption, Distribution, Metabolism, Excretion, and Toxicity) analysis platform.

That is, the AI-drug platform according to the present disclosure is configured to select active compounds (e.g., hit compounds), identify lead compounds among them, and then select drug candidates through drug response analysis, thereby performing the entire drug development process within an AI-based platform.

FIG. 2 illustrates the cloud service process of the AI-drug platform according to the present disclosure. As shown therein, the technology of the present disclosure provides all aspects of the drug discovery and development process, ranging from hit compound discovery, lead compound generation, and ADMET/PK to pharmacogenomics and biomarkers.

Furthermore, to operate the platform for each stage of new drug discovery, the AI-drug platform of the present disclosure applies three distinct AI systems: Generative AI system (GPT/BERT), Three-dimensional structure AI system (ED-CNN), and Molecular dynamics analysis system (Auto-MD simulation).

Concrete examples of the method for performing each of the automated active (hit) compound discovery platform, automated lead compound discovery platform, and automated drug response (ADMET: Absorption, Distribution, Metabolism, Excretion, and Toxicity) analysis platform by using the AI systems of the AI-drug platform include: DMC-PRE: Discovery of hit compounds based on three-dimensional structural information of protein-ligand interactions (applicant-defined technical term), GAP-Dock: Docking structure analysis of protein-ligand interactions based on central atom vectors (applicant-defined technical term), DMC-SCR: Prediction of optimized protein-compound binding conformations using a 3D-CNN trained model (applicant-defined technical term), LEAD-GEN: Generation of compound derivatives for a target protein (applicant-defined technical term), DMC-MD: Analysis of protein-compound binding stability using molecular dynamics simulation data (applicant-defined technical term), and 3bmGPT: Generative AI model trained on three-dimensional protein-compound interaction data (applicant-defined technical term).

Among these technologies, DMC-PRE and GAP-Dock are preliminary screening technologies applied in the automated hit compound discovery platform to identify hit compounds; DMC-SCR is applied after hit compounds are identified through the automated hit compound discovery platform and uses molecular dynamics analysis for in-depth screening; and LEAD-GEN is applied in the automated lead compound discovery platform for lead compound discovery.

DMC-MD is applied in the molecular dynamics analysis system to validate the binding stability of results obtained from the automated hit compound discovery platform, the automated lead compound discovery platform, and the automated drug response analysis platform, and 3bmGPT is applied in the generative AI system to select analyte compounds during the hit compound discovery process in the automated hit compound discovery platform.

Specifically, as illustrated in FIG. 3, the hit compound discovery process in the AI-drug platform according to the present disclosure proceeds as follows: the 3bmGPT model is used to select analyte compounds; the DMC-PRE and GAP-Dock technologies are applied for preliminary screening; the DMC-SCR technology is applied for in-depth screening; and the DMC-MD technology is applied to validate binding stability, ultimately deriving hit compounds.

Turing to the lead compound discovery process in the AI-drug platform according to the present disclosure, as illustrated in FIG. 4, the LEAD-GEN technology is applied to discover lead compounds, followed by the DMC-MD technology to validate binding stability, ultimately deriving lead compounds.

The technology of the present disclosure pertains to a compound derivative generation method for a target protein, which is applied to the aforementioned AI-drug platform (referred to as LEAD-GEN). The specific embodiments of this method will be described in detail below with reference to the drawings.

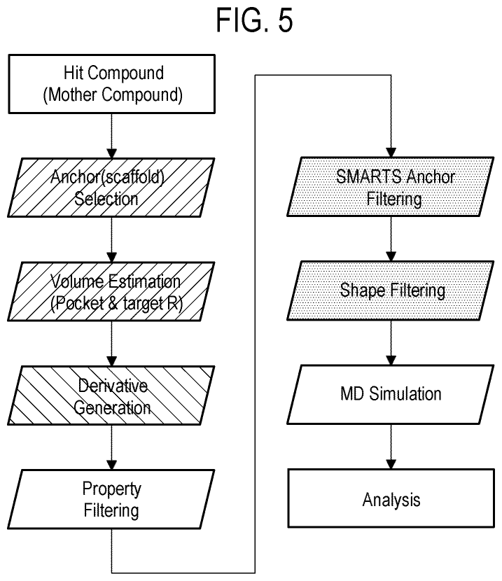

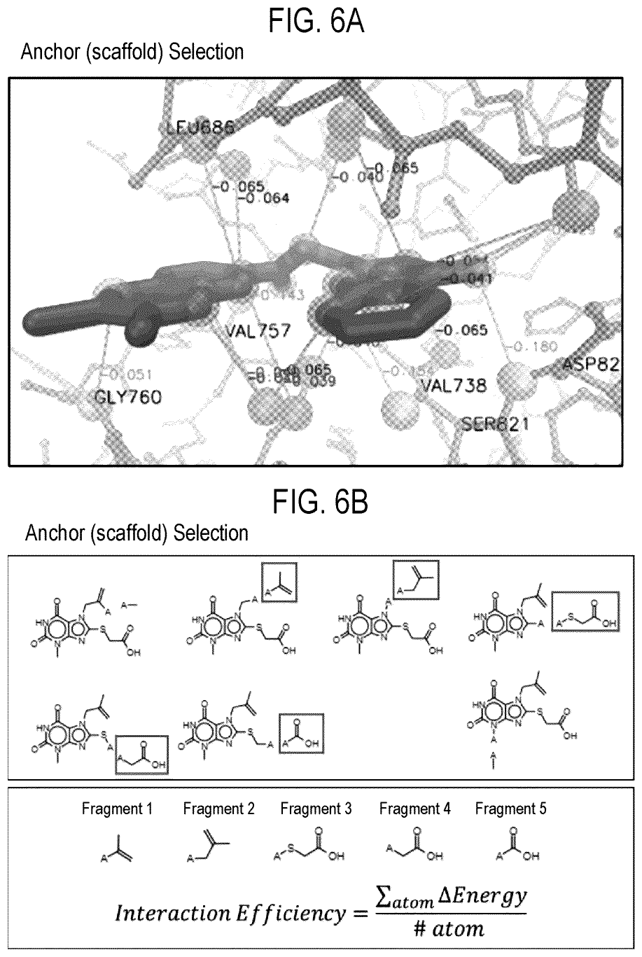

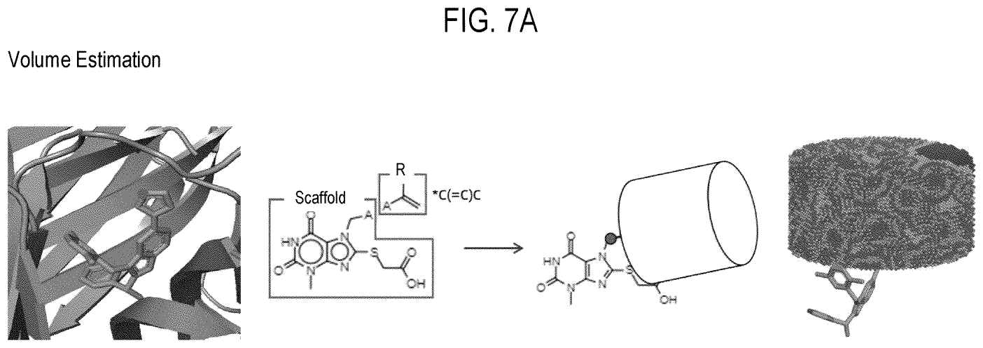

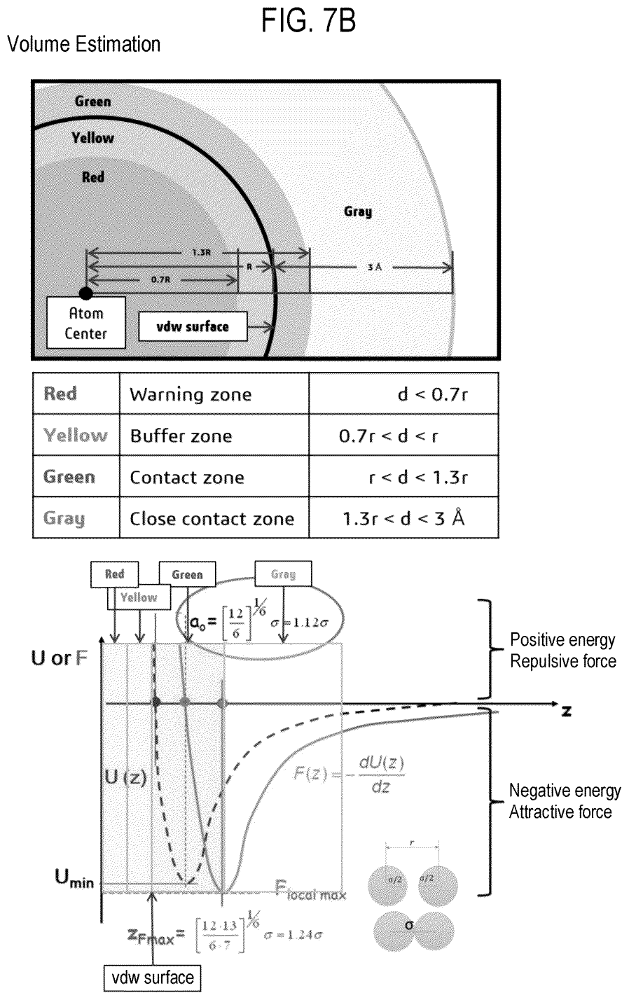

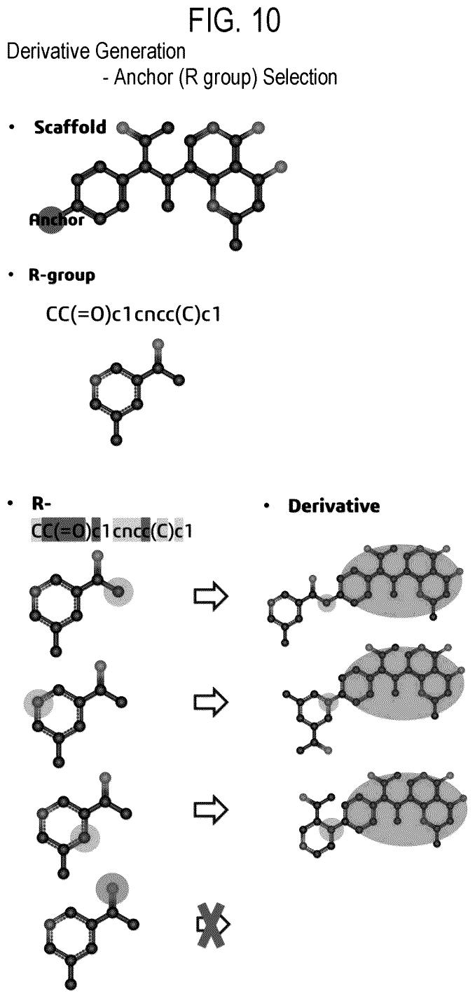

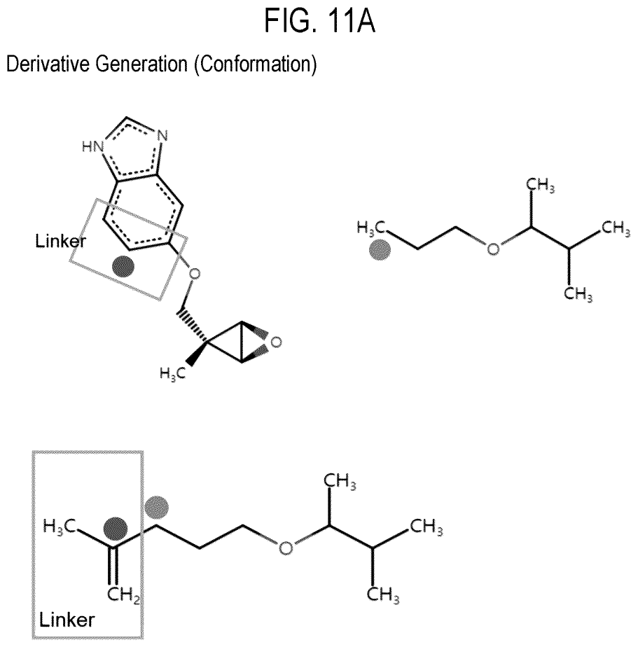

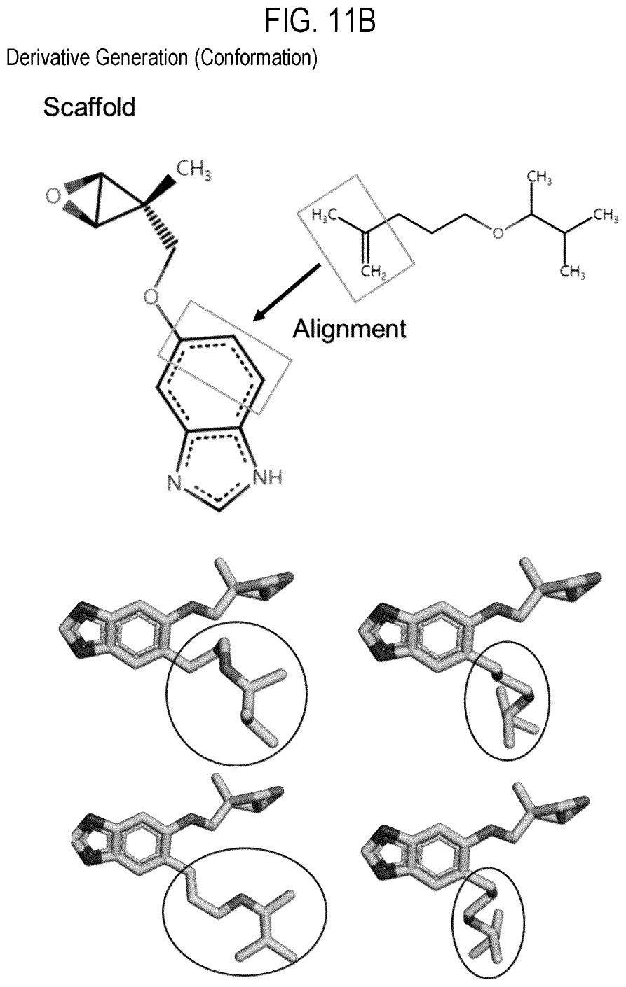

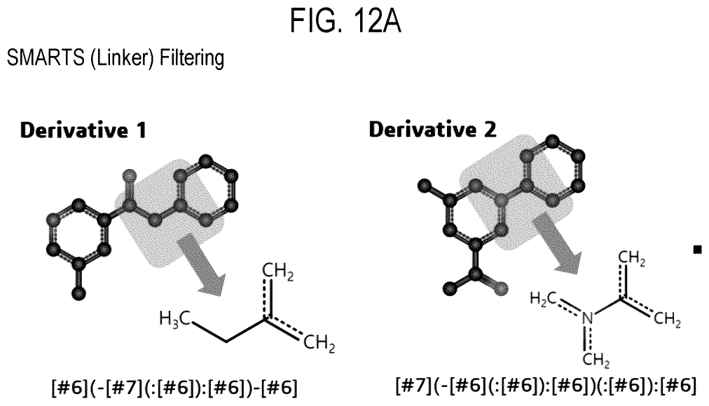

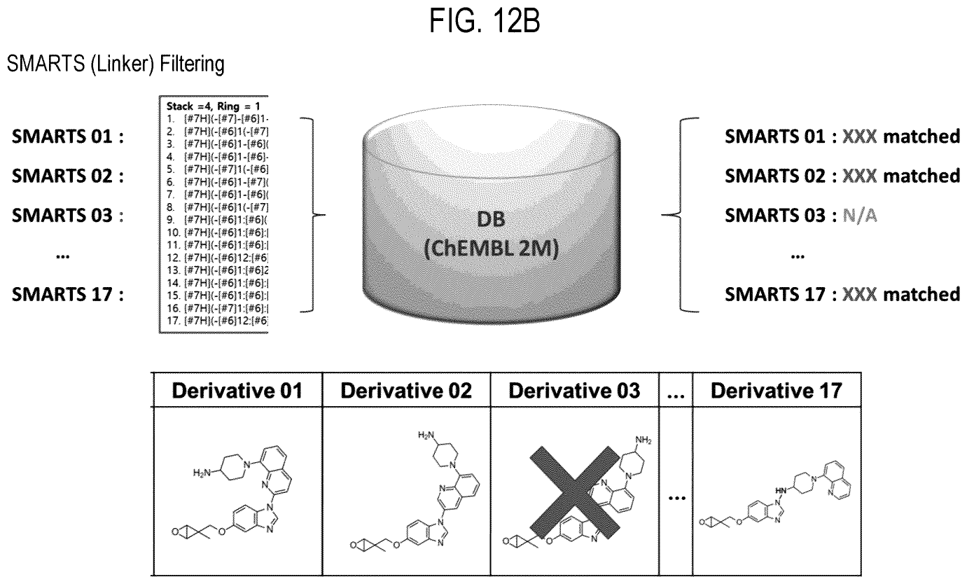

FIG. 5 is a flowchart illustrating the lead compound discovery method through derivative generation of a hit compound for a target protein according to the present disclosure. FIGS. 6A and 6B are conceptual diagrams illustrating a method for selecting the substitutable portion and scaffold (anchor molecule; anchor atom) in the derivative generation process according to the present disclosure. FIGS. 7A, 7B, 8, and 9 are conceptual diagrams illustrating the process of calculating the size of the target space within the target protein during the derivative generation process according to the present disclosure. FIGS. 10, 11A, and 11B are conceptual diagrams illustrating a method for selecting a substituent to generate a derivative in the derivative generation process according to the present disclosure. FIGS. 12A and 12B are an exemplary diagram illustrating a process for filtering substituents in the derivative generation process, where substituents are filtered by comparing the bonding moiety between the substituent and the scaffold as well as the binding conformation of the substituent with existing molecules.

The method for generating derivatives of a hit compound for a target protein according to the present disclosure primarily may include the steps of: (A) selecting a substitutable portion and a scaffold excluding the substitutable portion in the chemical structure of the hit compound; (B) setting a target space within the target protein, around the region where the selected substitutable portion of the hit compound binds; and (C) selecting a substituent that can replace the substitutable portion of the hit compound within the set target space of the target protein, and generating a derivative.

The method for generating derivatives of a hit compound for a target protein according to the present disclosure may be performed by a computational system.

In step (A), the substitutable portion in the chemical structure of the hit compound may be selected based on an indicator showing that it exhibits lower binding interaction compared to other chemical structures, as determined from the interaction profile between the hit compound and the target protein. The binding interaction may be calculated by sequentially cleaving each bond in the hit compound that interacts with the target protein, selecting a substitution candidate portion containing the atoms on the cleaved side, and deriving its value as the average binding energy of the constituent atoms with the target protein.

In the present disclosure, the substitution candidate portion may be selected through the steps of cleaving a bond (e.g., a single bond) within the hit compound and generating atomic fragments on both sides of the cleavage. The substitution candidate portion may include 1 to 12 atoms, and the derivative generation method according to the present disclosure may further include an additional step of filtering the substitution candidate portions based on the number of constituent atoms.

Specifically, as illustrated in FIG. 6A, when a hit compound, which is an active material, is bound to a target protein, the interaction profile (binding information) between the hit compound and the target protein is computed.

The interaction profile may be obtained using commercially available software or custom-developed software capable of extracting the necessary information, such as FEP+ or MMPBSA.

Next, as shown in FIG. 6B, with reference to the interaction profile between the hit compound the target protein, one of the bonds within the hit compound that interacts with the target protein is cleaved, generating atomic fragments on both sides of the cleavage.

This bond cleavage and fragment generation process is performed throughout the entire structure of the compound, resulting in twice the number of bonds as atomic fragments.

Among the generated atomic fragments, only those containing atoms within a predetermined range (e.g., 1 to 12 atoms) may be selected, while the remaining fragments may be excluded from analysis.

The purpose of selecting atomic fragments is to identify fragments with minimal impact on the binding interaction between the hit compound and the target protein, making them valuable as substitutable portions. When the selected atomic fragment contains too few atoms, it is less likely to result in a new compound upon substitution. Conversely, when the atomic fragment contains too many atoms, the chemical properties of the substituted compound may significantly deviate from the original hit compound.

To select the substitutable portion, the interaction efficiency of each atomic fragment may be calculated. Atomic fragments with low interaction efficiency may be considered weakly interacting chemical structures in terms of binding interaction strength and may therefore be regarded as substitutable portions. The remaining portion of the chemical structure, excluding the substitutable portion, is designated as the scaffold.

In the chemical structure of a hit compound that binds to a target protein, the anchor atom may be selected as an atom on the scaffold that is bound to the substitutable portion. That is, as for the selected atomic fragments, as illustrated in FIG. 6B, after calculating the interaction efficiency, the cleavage site of an atomic fragment with low binding interaction impact may be designated as an anchor.

The interaction efficiency may be calculated as the average binding energy of the atoms constituting the atomic fragment.

In step (B), the target space is derived as a space that includes the interaction region between the target protein and the hit compound (also referred to as the pocket space), which is later used for derivative generation.

Information regarding the binding pose of the target protein and the hit compound, as well as the shape of the interaction region, may be obtained from RCSB PDB, etc.

In step (B), the size of the target space within the target protein may be set so that it can accommodate the region of the hit compound that remains after excluding the selected substitutable portion, which belongs to the binding region of the target protein. Furthermore, in step (B), the target space may be determined by stepwise classifying the region of the target protein constituting the target space based on the interaction energy between the atoms of the target protein present in that region. The interaction energy may be classified into three to five levels, and the regions with relatively lower interaction energy among the classified levels may be clustered. Additionally, the clustering regions as the target space to be used for generating the hit compound derivative may be selected based on their proximity to the scaffold of the hit compound and the size of the clustering regions.

For example, the classification of interaction energy between atoms of the target protein may be performed by extracting the region of the target protein constituting the target space in the form of a cylindrical filter, setting marker points (dots) arranged at equal intervals in the cylindrical filter, and stepwise classifying the marker points based on the interaction energy between atoms of the target protein.

The selection of the clustered regions within the target space to be used for generating the hit compound derivative may be performed through the steps of: (B1) extracting the region of the target protein constituting the target space in the form of a cylindrical filter, setting marker points (dots) arranged at equal intervals within the cylindrical filter, and stepwise classifying the marker points based on the interaction energy between atoms of the target protein; and (B2) approaching the scaffold of the hit compound to the cylindrical filter, excluding regions in the cylindrical filter where the interaction energy exceeds a preset threshold, and then clustering the remaining marker points into spatial units; and optionally (B3) further selecting some of the clustered regions as the target space for derivative generation based on the proximity between marker points and the scaffold of the hit compound and the size of the clustered regions.

The clustering may be performed based on the density of regions where the interaction energy between atoms of the target protein is relatively low, or may utilize Gaussian Mixture Model (GMM) clustering, but with no limitations thereto.

Specifically, step (B) for deriving the target space (pocket space) within the target protein may involve calculating the internal space that influences the interaction when the scaffold with the substitutable portion removed therefrom binds within the interaction region of the target protein, as illustrated in FIGS. 7A, 7B, 8, and 9.

To this end, as illustrated in FIGS. 7A and 7B, a cylindrical region with a preset size (length 10 Å, radius 10 Å) is extracted around the substitutable portion or anchor region of the hit compound relative to the target protein, generating a cylindrical region filter.

The size of the region filter may be adjusted depending on the structure of the scaffold and the computational resources available in the system. However, it is preferably set to sufficiently cover the interaction region of the scaffold with the target protein.

Furthermore, marker points (dots) are arranged at equal intervals within the cylindrical region filter, and these marker points are classified based on interaction energy between atoms of the target protein. As illustrated in FIG. 7A, the marker points within the cylindrical region filter are color-coded based on interaction energy between atoms of the target protein.

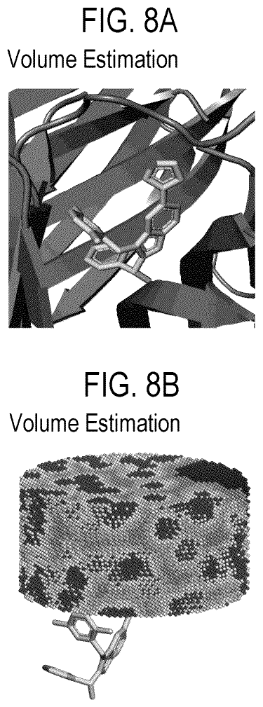

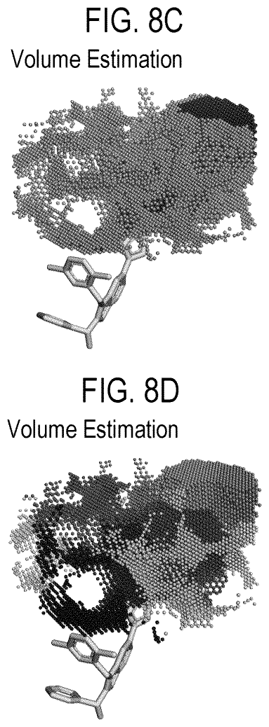

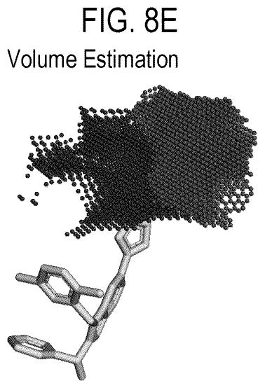

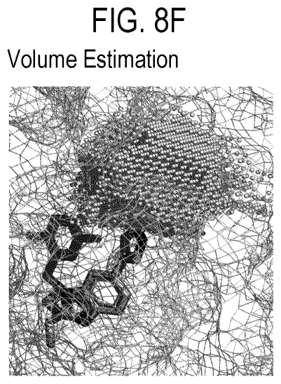

As illustrated in FIGS. 8A and 8B, the anchor region of the scaffold was subsequently aligned and positioned to approach the anchor region of the cylindrical region filter. At this time, the binding axis direction of the scaffold may be maintained in its original binding orientation.

Next, as illustrated in FIG. 8C, regions with high interaction energy between atoms of the target protein (red zone, yellow zone, and green zone) were excluded from the cylindrical region filter, leaving only the marker points corresponding to regions with low interaction energy (gray zone, dark gray zone).

The remaining marker points were then clustered into spatial units, as shown in FIG. 8D (e.g., GMM clustering). Among the clustered regions, only some were selected based on their proximity to the scaffold anchor and the size of the clustered regions. In a specific embodiment, selection was made of the two largest clustered regions connected to the scaffold anchor (FIG. 8E).

As illustrated in FIG. 8F, the selected clustered regions were derived as the target space (also referred to as the target volume or pocket space) within the target protein. The reason for deriving the target space within the target protein is to select an appropriate substituent (R-group; Replace atom group) to replace the substitutable portion, based on the size and shape of the target volume.

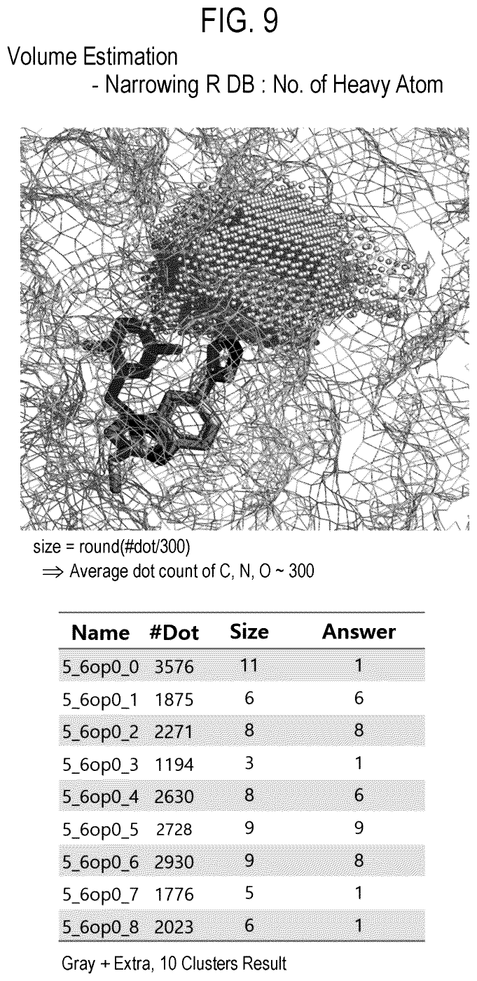

FIG. 9 illustrates an example where the target space (target volume) within the actual target protein was derived using the aforementioned method.

In the embodiment shown in FIG. 9, nine anchor points were derived for compound 6op0, and upon analysis, the results were found to match the actual number of ligand atoms in the compound. Specifically, to represent the size of the target space, the number of marker points included in the target space was divided by 300, which corresponds to the average volume typically occupied by a heavy atom (C, N, O) in a ligand. The results confirmed that the calculated number of atoms within the target space corresponded to the actual number of atoms in the ligand of the compound.

Step (C), which is a step of generating derivatives, is carried out by selecting a substituent from a substituent group database that can be accommodated within the target space set in step (B) and substituting same for the substitutable portion of the hit compound selected in step (A).

That is, as illustrated in FIG. 10, step (C) of generating derivatives involves selecting a substituent that fits the size of the target space derived in step (B) within the target protein.

Herein, the term “substituent” refers to an atomic group that replaces the substitutable portion, which is an atomic fragment removed from the scaffold. The substituent is selected from a database containing various atomic compositions. After selection, the substituent is bound to the anchor of the scaffold to generate a derivative.

In this regard, derivatives with multiple different binding conformations may be generated for the same substituent by varying the binding position of the substituent that binds to the scaffold. As illustrated in FIG. 10, even with the same substituent, different derivatives may be generated by varying the position where the substituent binds to the anchor atom of the scaffold.

Additionally, for a substituent with the same binding conformation, derivatives having multiple different binding poses may be generated by modifying the binding angle between the substituent and the scaffold of the hit compound. As illustrated in FIGS. 12A and 12B, even when the binding conformation remains the same, multiple derivatives may be generated by varying the binding pose (binding angle) between the substituent and the scaffold.

In this case, modifying the binding poses between the substituent and the scaffold to generate various derivatives having binding poses can be achieved by extracting the linker of the anchor region of the target protein, as illustrated in FIGS. 12A and 12B.

Herein, the linker refers to the region proximate to the anchor, which is extracted by selecting a preset number of bonded atoms from the anchor. In the embodiment illustrated in FIGS. 12A and 12B, the linker was extracted up to three bonded atoms from the anchor atom.

By diversifying the binding portion and modifying the binding pose of the substituent, multiple derivatives with varied binding conformations can be generated.

The method for generating derivatives of a hit compound for a target protein according to the present disclosure may further include step (D) of filtering the generated derivatives.

Various criteria may be used to filter the generated derivatives. First, derivatives with low feasibility for actual synthesis or existence may be excluded based on real compound structure data. Additionally, filtering may consider the binding conformation of the substituent, particularly to account for atomic clashes that may occur within the target space. Here, atomic clashes refer to collisions between atoms of the derivative and atoms of the target protein.

The filtering process of the generated derivatives in the present disclosure may be categorized into two primary criteria. The first criterion is filtering binding poses between the binding site and substituents against a real compound structure database to determine feasibility (also called linker filtering) and the second criterion is binding pose filtering (also called shape filtering): filtering by evaluating atomic clashes that occur within the target space when the substituent is bound to the cylindrical region filter based on its binding pose.

As illustrated in FIGS. 12A and 12B, linker filtering involves comparing various binding conformations of the linker and substituent with actual compound data from a chemical database (e.g., ChEMBL) to selectively identify viable structures.

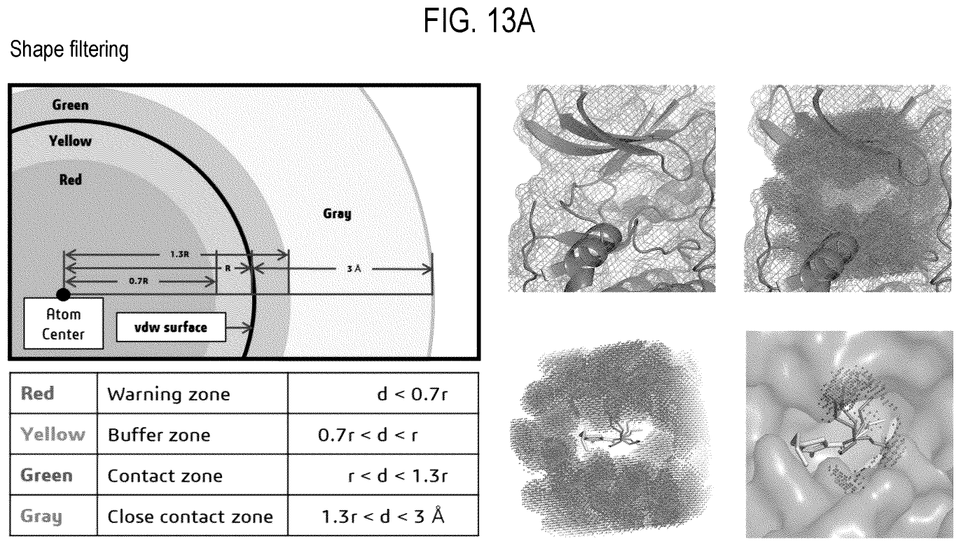

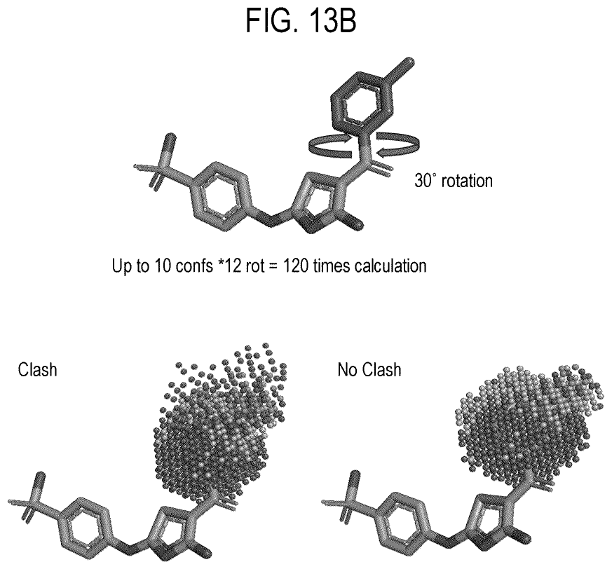

Specifically, the binding conformation of the linker and substituent is searched within a compound database, and their binding poses are analyzed. Only derivatives with binding poses that exist in actual compounds are selected. During this process, as illustrated in FIGS. 12A and 12B, the binding pose of a specific binding region may be determined based on the SMARTS pattern-matching system, which is used for molecular pattern description. Additionally, as illustrated in FIGS. 13A and 13B, the generated derivatives in the compound derivative generation process may be filtered based on whether atomic clashes occur within the target space or based on the degree of atomic clashes. Atomic clashes refer to collisions between atoms of the derivative and atoms of the target protein.

Below, a better understanding of the present disclosure may be obtained through the following examples which are set forth to illustrate the configurations and advantages of the present disclosure, but are not to construed to limit the scope and extent of the present disclosure.

### Example 1 Analysis of Hit Compound Binding to Target Protein

To verify the effectiveness of the method for generating derivatives of a hit compound for a target protein, selection was made of an exemplary target protein and a hit compound binding thereto, and the method of the present disclosure was applied.

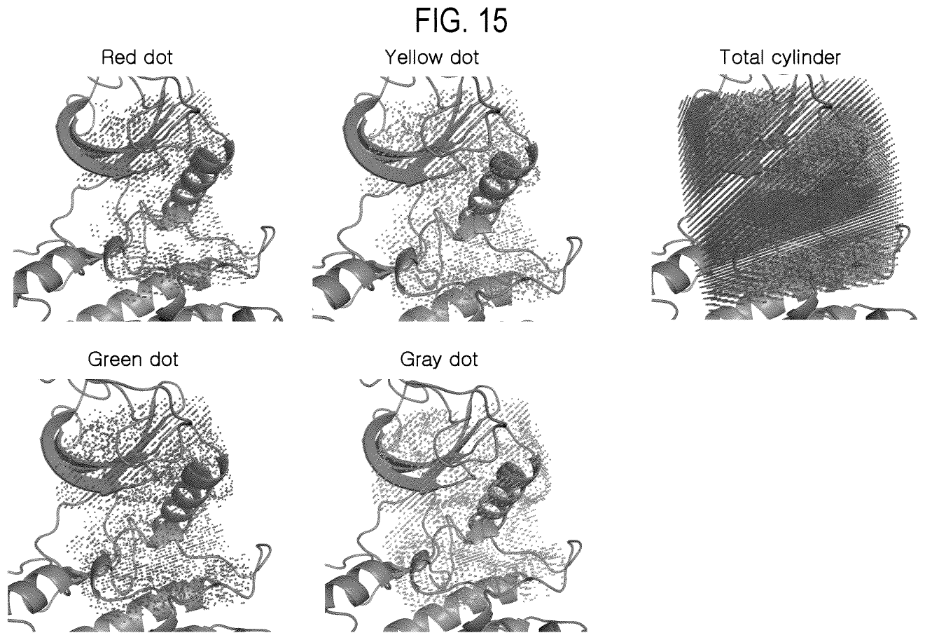

In this example, the target protein was a protein with kinase activity (hereinafter referred to as protein A), and the hit compound binds to the ATP-binding pocket where the kinase interacts with ATP. Specifically, protein A has a structure comprising: five beta-sheets (B-sheets) at the top, a central cavity, and respective alpha-helices (a-helices) at the left, right, and bottom surrounding the central cavity. This structure corresponds to common and similar structures among kinase proteins that contain an ATP-binding pocket. The specific structure can be confirmed in the modeling diagrams of FIGS. 15 and 17.

The inventors previously identified a compound that binds to protein A, specifically C₂₁H₂₀N₆O₂S (SMILES: O=C (NC1=CC=CC(C2=NNC(NC(C3=NC(C=CC=N4)=C4S3)=O)=C2)=C1)C(C)(C)C), which was designated as Hit compound X. Hit compound X was used as the mother compound for the derivative generation process.

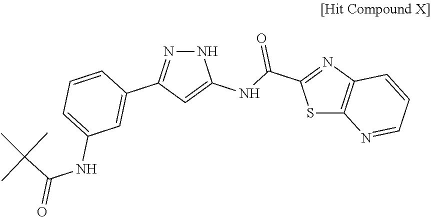 [SMILES: CC(C)(C)C(=O)Nc1cccc(-c2cc(NC(=O)c3nc4cccnc4s3)[nH]n2)c1]

The structural information of protein A and hit compound X was input into a binding interaction calculation program to analyze the interaction profile, which includes data on whether and how strongly two atoms interact.

Specifically, using the program, the interaction profile between hit compound X and protein A was calculated. Then, each single bond (o-bond) within hit compound X that interacts with protein A was individually cleaved, generating atomic fragments on both sides of the cleavage. The atomic fragment closest to protein A was selected as a substitution candidate portion. Based on the interaction between protein A and hit compound X, a substitutable portion was selected from the substitution candidate portion. Herein, the binding interaction refers to interaction efficiency, a well-established metric in this technical field.

The atomic fragments generated as substitution candidate portions were first filtered based on the number of constituent atoms. Only atomic fragments consisting of 1 to 12 atoms were retained, and the rest were filtered out. Among the filtered atomic fragments, the substitutable portion was selected based on low binding interaction. Specifically, the interaction efficiency of each filtered atomic fragment was calculated. Interaction efficiency is calculated as the average binding energy of the atoms constituting the atomic fragment. The equation used for calculating interaction efficiency is as follows:

$$Interaction Efficiency = \frac{{\sum }_{atom} Δ Energy}{\# atom} \qquad [ Equation ]$$

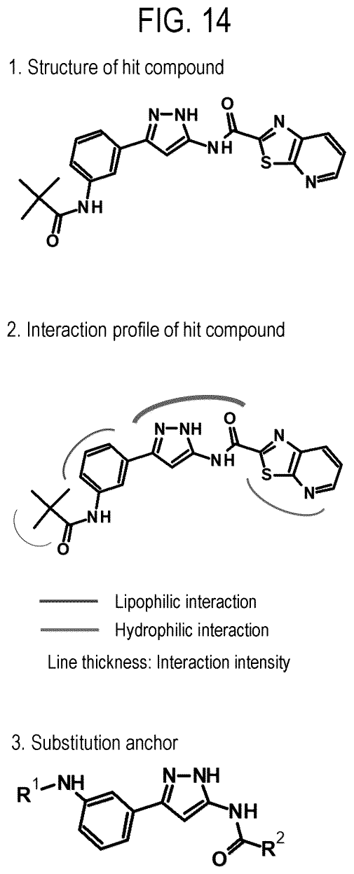

As illustrated in FIG. 14 (2: Interaction Profile of Hit compound), the interaction profile of hit compound X was analyzed to identify the regions interacting with protein A and the strength of these interactions.

Regions within the structure of hit compound X that exhibit weak interactions with protein A correspond to atomic fragments with low interaction efficiency. These regions also correlate with chemical structures that exhibit lower binding interaction in the interaction profile.

Atomic fragments with low interaction efficiency were selected as substitutable portions.

The atom in the scaffold to which the substitutable portion was bound was designated as the anchor atom.

Thus, both terminal ends, which corresponded to the weak interaction regions of the compound, were designated as substitutable portions (R¹ and R² in FIG. 14, 3: Substitution Anchor) while the remaining portion was designated as the scaffold (excluding R¹ and R² in Section 3 of FIG. 14). The atoms in the scaffold which bind to R¹ and R² in Section 3 of FIG. 14, that is, the nitrogen (N) atom and carbon (C) atom in the scaffold, were designated as anchor atoms.

### Example 2 Setting Target Space within Target Protein

The target space within the target protein that interacts with the substitutable portion of the hit compound X selected in Example 1 was established for use in a subsequent derivative generation process. In this regard, the interaction region within protein A where hit compound X binds was analyzed.

Specifically, within protein A, a binding region around the anchor was extracted, and a cylindrical region filter was generated. To adequately reflect the environment influencing the interaction between hit compound X and protein A, a cylindrical region with a radius of 10 Å and a height of 10 Å was extracted (FIG. 15).

Within the extracted region filter, marker points (dots) were arranged at equal intervals, and these marker points were classified based on interaction energy between atoms of protein A. Specifically, as illustrated in FIG. 7B, the marker points were classified based on the van der Waals (vdW) radius (r) calculation formula: red dots (red zone; clash region) for marker points within 0.7r, representing atomic clashes; yellow dots (yellow zone; buffer region) for Marker points within approximately 0.7r to 1.0r, representing buffer zones; green dots (green zone; contact region) for marker points within approximately 1.0r to 1.3r, representing contact areas; and gray dots (gray zone; close region) for marker points within approximately 1.3r to 3 Å, representing nearby zones.

Among the colored zones, the gray zone with low interaction energy between atoms of the target protein was considered the most likely pocket region where a compound could bind, as it provides sufficient space for derivative formation.

The anchor atom of the scaffold in hit compound X from which the substitutable portion was removed was positioned to approach the anchor region of the cylindrical region filter. Among the marker dots, high-interaction energy marker dots were excluded from the cylindrical region filter. Then, the remaining marker points within the filter were clustered into spatial units using Gaussian Mixture Model (GMM) clustering. Based on proximity to the anchor atom and cluster size, only two clustered regions were selected. These two clustered regions were designated as the target space (pocket space; target volume) within the target protein, and the size of the target space was calculated.

### Example 3 Generation of Derivatives Through Substituent Selection

The substitutable portion of the hit compound selected in Example 1 was replaced with a substituent that could be accommodated within the target space set in Example 2, thereby generating derivatives. This was accomplished by selecting a substituent from a substituent group database and substituting same into the hit compound.

Specifically, a substituent was selected, and the optimal binding conformation between the substituent and the anchor atom was analyzed. Once the binding conformation was determined, multiple derivatives were generated by checking diverse binding poses at various binding angles between the substituent and the scaffold.

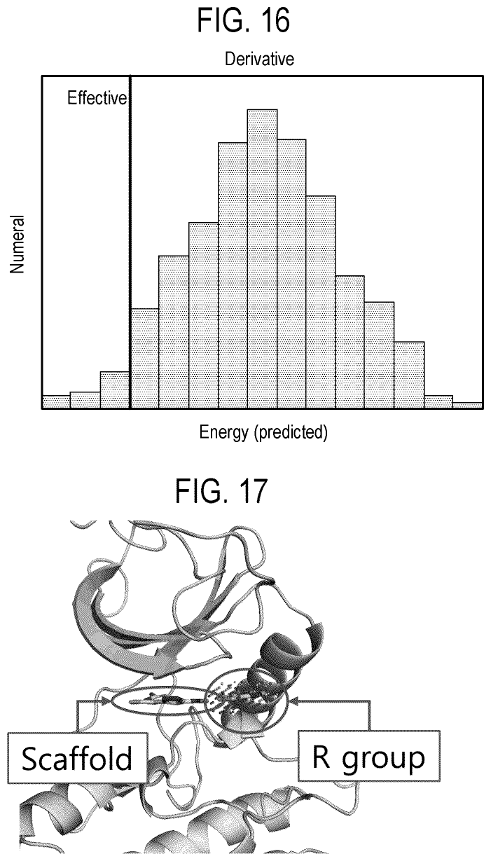

The binding energy of each generated derivative was calculated. The derivatives with binding energy in the lowest 5% range were selected as stable derivatives (see FIG. 16). Among them, derivatives with high feasibility for synthesis and real compound existence were selected through comparison with existing compounds. The selected derivatives were then synthesized as actual compounds. As confirmed in Table 2, the specific embodiments of the present disclosure demonstrated that in silico-generated derivatives could be successfully synthesized as real compounds.

### Example 4 Verification of Target Protein Inhibition by Synthesized Hit Compound Derivatives

The hit compound derivatives synthesized as real compounds through Example 3 were evaluated for ability to inhibit the activity of protein A (a kinase). Additionally, their inhibitory effect was compared to that of the original hit compound to determine whether they exhibited similar or superior efficacy.

Specifically, 20 u M of protein A was incubated along with 8 mM MOPS (pH 7.0), 0.2 mM EDTA, 10 mM magnesium acetate, 10 μM [gamma-33P]-ATP, and 10 μM YRRAAVPPSPSLSRHSSPHQSEDEEE, which is a phosphorylation target peptide. After incubation at room temperature for 40 minutes, the reaction was terminated by adding phosphoric acid to a final concentration of 0.5%. The reaction mixture was spotted onto a filter, then washed four times with 0.425% phosphoric acid for 4 minutes each. After washing once with methanol, the sample was dried and measured for radioactivity (scintillation counting) to assess phosphorylation (i.e., YRRAAVPPSPSLSRHSSPHQS(p)EDEEE peptide detection).

The results were compiled into Tables 2 and 3. Specifically, Table 2 summarizes the structure, IC₅₀, and pIC50 values of the derivatively in-real synthesized in Example 3, indicating their inhibitory effects on protein A while Table 3 summarizes the predicted binding energy of each derivative with protein A and their pIC50 values.

TABLE 2

| Cpd. | No. | SMILES | Chem. Formula | IC50 | pIC50 |
|---|---|---|---|---|---|
| D0065 | 1 | O═C(NC1═CC═CC(C2═NNC(NC(C3═NC | C₂₁H₂₀N₆O₂S | 0.119 | 6.924 |
| | | (C═CC═N4)═C4S3)═O)═C2)═C1)C(C)(C)C | | | |
| D0083 | 2 | O═C(NC1═CC(C2═CC(NS(N3CCOCC3) | C₂₁H₂₀N₆O₄S₂ | 0.129 | 6.889 |
| | | (═O)═O)═CC═C2)═NN1)C4═CC═CC5═C4SC═N5 | | | |
| D0135 | 3 | C#CC(NC1═CC═CC(C2═NNC(NC(C3═CC4═C | C₂₁H₁₄N₄O₄ | 0.063 | 7.201 |
| | | (O3)C═C(O)C═C4)═O)═C2)═C1)═O | | | |
| D0025 | 4 | O═C(NC1═CC(C2═CC(NS(N3CCOCC3) | C₂₀H₁₉N₇O₄S₂ | 0.062 | 7.208 |
| | | (═O)═O)═CC═C2)═NN1)C4═CC5═C(S4)N═CC═N5 | | | |
| D0137 | 5 | C#CC(NC1═CC═CC(C2═NNC(NC(C3═CC4═C | C₁₉H₁₂N₆O₃S | 0.040 | 7.398 |
| | | (S3)N═C(O)C═N4)═O)═C2)═C1)═O | | | |
| D1010 | 6 | CC#CC(NC1═CC(C2═NNC(NC(C3═CC4═C | C₂₁H₁₉N₅O₂S | 0.031 | 7.509 |
| | | (S3)CCNC4)═O)═C2)═CC═C1)═O | | | |
| D0069 | 7 | O═C(NC1═CC═CC(C2═NNC(NC(C3═CC4═C | C₂₃H₁₈N₄O₄ | 0.030 | 7.523 |
| | | (C═CC(OC)═C4)O3)═O)═C2)═C1)C#CC | | | |
| D0054 | 8 | C#CC(NC1═CC═CC(C2═NNC(NC | C₁₉H₁₂N₆O₃ | 0.026 | 7.585 |
| | | (C3═CC4═NON═C4C═C3)═O)═C2)═C1)═O | | | |
| D0139 | 9 | C#CC(NC1═CC═CC(C2═NNC(NC | C₂₁H₁₄N₄O₄ | 0.022 | 7.658 |
| | | (C3═CC4═C(O3)C═C(O)C═C4)═O)═C2)═C1)═O | | | |
| D1008 | 10 | CC#CC(NC1═CC(C2═NNC(NC(C3═CC4═C | C₂₁H₁₈N₄O₃S | 0.019 | 7.721 |
| | | (S3)CCOC4)═O)═C2)═CC═C1)═O | | | |
| D0101 | 11 | C#CC(NC1═CC═CC(C2═NNC(NC(C3═CC4═C | C₁₉H₁₃ClN₆O₂S | 0.013 | 7.886 |
| | | (N═CC═N4)S3)═O)═C2)═C1)═O•Cl | | | |
| DM010 | 12 | C#CC(═O)Nc4cccc(c3cc(NC(═O)c2cc1ncc | C₂₀H₁₃N₇O₃S | 0.010 | 8.000 |
| | | (C(N)═O)nc1s2)[nH]n3)c4 | | | |
| D1011 | 13 | CC#CC(NC1═CC(C2═NNC(NC(C3═CC | C₂₁H₁₇N₅O₃S | 0.008 | 8.097 |
| | | (CN4)═C(S3)CC4═O)═O)═C2)═CC═C1)═O | | | |
| DM009 | 14 | C#CC(═O)Nc4cccc(c3cc(NC(═O)c2cc1ncc | C₂₀H₁₂N₆O₄S | 0.007 | 8.155 |
| | | (C(═O)O)nc1s2)[nH]n3)c4 | | | |
| D0011 | 15 | O═C(C1═CC(N═CC═N2)═C2S1)NC3═CC | C₁₉H₁₂N₆O₂S | 0.013 | 7.886 |
| | | (C4═CC(NC(C#C)═O)═CC═C4)═NN3 | | | |
| D0157 | 16 | C#CC(NC1═CC═CC(C2═NNC(NC(C3═CC4═C | C₂₀H₁₆N₄O₃S | 0.004 | 8.398 |
| | | (CCOC4)S3)═O)═C2)═C1)═O | | | |

TABLE 3

| | | | pIC₅₀ | Energy |
|---|---|---|---|---|
| Compound | R1 | R2 | (experimental) | (prediction) |
| D0065 (Hit) | 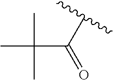 [SMILES: *C(=O)C(C)(C)C] | 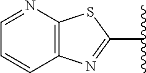 [SMILES: *c1nc2cccnc2s1] | −6.924 | −25.159 |
| D0083 | 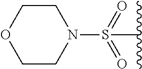 [SMILES: *S(=O)(=O)N1CCOCC1] | 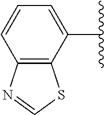 [SMILES: *c1cccc2ncsc12] | −6.889 | −33.281 |
| D0135 | 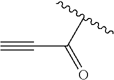 [SMILES: *C(=O)C#C] | 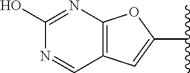 [SMILES: *c1cc2cnc(O)nc2o1] | −7.201 | −25.209 |
| D0025 | 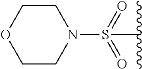 [SMILES: *S(=O)(=O)N1CCOCC1] | 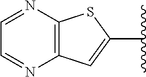 [SMILES: *c1cc2nccnc2s1] | −7.208 | −21.291 |
| D0137 | 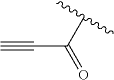 [SMILES: *C(=O)C#C] | 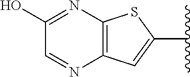 [SMILES: *c1cc2ncc(O)nc2s1] | −7.398 | −23.401 |
| D1010 | 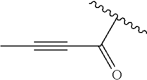 [SMILES: *C(=O)C#CC] | 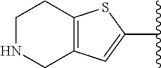 [SMILES: *c1cc2c(s1)CCNC2] | −7.509 | −28.555 |
| D0069 | 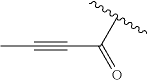 [SMILES: *C(=O)C#CC] | 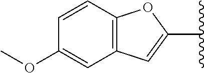 [SMILES: *c1cc2cc(OC)ccc2o1] | −7.523 | −28.815 |
| D0054 | 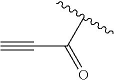 [SMILES: *C(=O)C#C] | 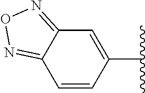 [SMILES: *c1ccc2nonc2c1] | −7.585 | −23.816 |
| D0139 | 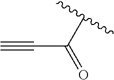 [SMILES: *C(=O)C#C] | 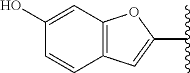 [SMILES: *c1cc2ccc(O)cc2o1] | −7.658 | −23.271 |
| D1008 | 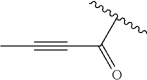 [SMILES: *C(=O)C#CC] | 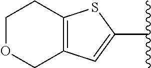 [SMILES: *c1cc2c(s1)CCOC2] | −7.721 | −26.162 |
| D0101 | 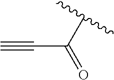 [SMILES: *C(=O)C#C] | 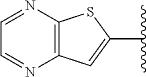 [SMILES: *c1cc2nccnc2s1] | −7.886 | −21.364 |
| DM010 | 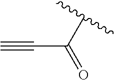 [SMILES: *C(=O)C#C] | 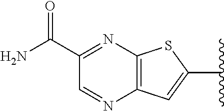 [SMILES: *c1cc2ncc(C(N)=O)nc2s1] | −8.000 | −29.596 |
| D1011 | 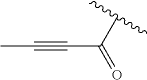 [SMILES: *C(=O)C#CC] | 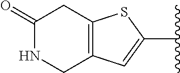 [SMILES: *c1cc2c(s1)CC(=O)NC2] | −8.097 | −21.176 |
| DM009 | 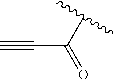 [SMILES: *C(=O)C#C] | 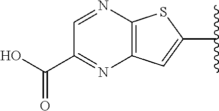 [SMILES: *c1cc2nc(C(=O)O)cnc2s1] | −8.155 | −21.032 |
| D0011 | 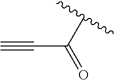 [SMILES: *C(=O)C#C] | 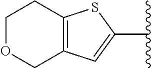 [SMILES: *c1cc2c(s1)CCOC2] | −7.886 | −23.821 |
| D0157 | 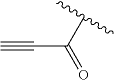 [SMILES: *C(=O)C#C] | 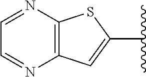 [SMILES: *c1cc2nccnc2s1] | −8.398 | −21.470 |

As shown in Tables 2 and 3, the pIC50 values of each derivative were measured. Many derivatives exhibited similar or superior inhibitory effects on protein A compared to hit compound X, as indicated by lower pIC50 values. Notably, as illustrated in FIG. 17, the derivative D0157 exhibited inhibition superior to the original hit compound by approximately e2-fold (log squared level).

As a result of modeling, the binding pose at which the derivative with a derivative predicted to be optima binds to protein A was expected to be consistent with the model illustrated in FIG. 17.

From the foregoing, a skilled person in the art to which the present disclosure pertains will be able to understand that the present disclosure may be embodied in other specific forms without modifying the technical concepts or essential characteristics of the present disclosure. In this regard, the exemplary embodiments disclosed herein are only for illustrative purposes and should not be construed as limiting the scope of the present disclosure. On the contrary, the present disclosure is intended to cover not only the exemplary embodiments but also various alternatives, modifications, equivalents, and other embodiments that may be included within the spirit and scope of the present disclosure as defined by the appended claims.

### BEST MODE FOR CARRYING OUT THE INVENTION

The best mode for carrying out the invention has been described above.

### INDUSTRIAL APPLICABILITY

The present disclosure can be applied to the new drug discovery process for building an AI-drug platform.

What is claimed is:

1. A method for generating a hit compound derivative from a hit compound for a target protein, the method comprising:
(A) selecting a substitutable portion and a scaffold excluding the substitutable portion in a chemical structure of the hit compound;
(B) setting a target space within the target protein, around a region where the selected substitutable portion of the hit compound binds; and
(C) selecting a substituent that can replace the substitutable portion of the hit compound within the set target space of the target protein, and generating the hit compound derivative.

2. The method of claim 1, wherein the method is for constructing an artificial intelligence (AI)-drug platform.

3. The method of claim 1, wherein the substitutable portion in the chemical structure of the hit compound in step (A) is selected based on an indicator showing lower binding interaction compared to other chemical structures, as determined from an interaction profile between the hit compound and the target protein.

4. The method of claim 3, wherein the binding interaction is determined by: cleaving individual bonds within the hit compound that interact with the target protein, selecting a substitution candidate portion that includes atoms at the cleaved site for each cleaved bond, and calculating an average binding energy between each atom constituting the substitution candidate portion and the target protein.

5. The method of claim 4, wherein the substitution candidate portion comprises 1 to 12 atoms.

6. The method of claim 5, further comprising filtering the substitution candidate portion based on the number of constituent atoms.

7. The method of claim 1, wherein a size of the target space within the target protein in step (B) is set to accommodate the selected substitutable portion of the hit compound in the chemical structure of the hit compound binding to the target protein.

8. The method of claim 1, wherein the setting in step (B) comprises determining the region of the target protein constituting the target space within the target protein by stepwise classification based on interaction energy between atoms of the target protein present in the region.

9. The method of claim 8, wherein the interaction energy is classified into three to five levels.

10. The method of claim 8, wherein regions with relatively lower level of the interaction energy are clustered, and the clustered regions of the target space to be used for generating the hit compound derivative are further selected based on proximity to the scaffold of the hit compound and size of the clustered regions.

11. The method of claim 8, wherein the classification of interaction energy between atoms of the target protein in step (B) is performed by: extracting the region of the target protein constituting the target space as a spatial filter, setting marker points (dots) arranged at equal intervals in the spatial filter, and stepwise classifying the marker points based on interaction energy between atoms of the target protein.

12. The method of claim 11, wherein the spatial filter is a spherical, rectangular, cylindrical, or amorphous filter.

13. The method of claim 10, wherein the selection of clustered regions of the target space to be used for generating the hit compound derivative in step (B) comprises:
(B1) extracting the region of the target protein constituting the target space as a cylindrical filter form, setting marker points (dots) arranged at equal intervals in the cylinder filter, and stepwise classifying the marker points based on the interaction energy between atoms of the target protein; and
(B2) approaching the scaffold of the hit compound to the cylindrical filter, excluding regions where interaction energy exceeds a preset threshold from the cylindrical filter, and then clustering the remaining marker points into spatial units.

14. The method of claim 13, wherein the selection of clustered regions of the target space to be used for generating the hit compound derivative in step (B) further comprises (B3) selecting a part of the clustered regions as the target space to be used for generating the hit compound derivative, based on proximity between marker points in the clustered regions and the size of the clustered regions.

15. The method of claim 10, wherein clustering is performed based on the density of regions with relatively lower level of the interaction energy, or by using Gaussian Mixture Model (GMM) clustering.

16. The method of claim 1, wherein step (C) is performed by replacing the substitutable portion that binds to the scaffold of the hit compound, selected in step (A), with a substituent selected from a substituent group database, which can be accommodated within the target space of the target protein set in step (B).

17. The method of claim 1, wherein the substitutable portion in the chemical structure of the hit compound in step (A) is selected based on an indicator showing lower lever of the binding interaction compared to other chemical structures, as determined from the interaction profile between the hit compound and the target protein,
wherein the selection of the target space to be used for generating the hit compound derivative of the target protein in step (B) is performed by the steps of:
(B1) extracting the region of the target protein constituting the target space as a cylindrical filter form, setting marker points (dots) arranged at equal intervals in the cylindrical filter, and stepwise classifying the marker points based on the interaction energy between atoms of the target protein,
(B2) approaching the scaffold of the hit compound to the cylindrical filter, excluding regions where the interaction energy exceeds a preset threshold from the cylindrical filter, and then clustering the remaining marker points into spatial units, and
(B3) selecting a portion of the clustered regions based on the proximity between marker points (dots) in the clustered regions and the size of the clustered regions, and
wherein the derivative generation in step (C) is performed by selecting substituents that can be accommodated within the target space selected in step (B).

18. The method of claim 17, wherein
(i) the derivative having multiple different binding conformations is generated for the same substituent by varying the binding position within the substituent that binds to the scaffold of the hit compound;
(ii) the derivative having multiple different binding poses is generated for the substituents with the same binding conformation by varying the binding between the substituents and the scaffold of the hit compound; or
(iii) the derivative having multiple different binding poses is generated for the substituents with the same binding conformation by varying the binding angle between the substituents and the scaffold of the hit compound.

19. The method of claim 17, wherein the binding interaction is determined by: cleaving individual bonds within the hit compound that interact with the target protein, selecting a substitution candidate portion that includes atoms at the cleaved site, and calculating average binding energy between each atom constituting the substitution candidate portion and the target protein.

20. The method of claim 1, further comprising (D) filtering the generated derivatives.

21. The method of claim 18, further comprising (D) filtering the generated derivatives, wherein step (D) comprises: at least one or more of
(i) filtering the binding angle between the substituent and the scaffold of the hit compound compared with a real compound database; and
(ii) filtering the derivatives based on degrees of atomic clash between the derivative and the target protein atoms, which occurs within the target space set in the derivatives in step (B), according to the binding pose of the substituents.
